(19) United States
(12) Patent Application Publication
(10) Pub. No.: US 2025/0336220 A1
(43) Pub. Date: Oct. 30, 2025

(54) MICROCODES

(71) Applicant: Georgia Tech Research Corporation, Atlanta, GA (US)

(72) Inventors: Ali Fatih Sarioglu, Atlanta, GA (US); Aref Valipour, Atlanta, GA (US); Jungmin Ha, Atlanta, GA (US)

(21) Appl. No.: 19/188,615

(22) Filed: Apr. 24, 2025

Related U.S. Application Data
(60) Provisional application No. 63/638,153, filed on Apr. 24, 2024.

(51) Int. Cl. G06V 20/69; C12M 1/12; C12M 1/32; C12M 1/34; H04N 7/18; H04N 23/63

(52) U.S. Cl. CPC G06V 20/693; C12M 23/12; C12M 25/02; C12M 41/36; H04N 7/185; H04N 23/631

(57) ABSTRACT

Disclosed herein are apparatuses, systems, and methods for measuring, viewing, identifying, or inspecting a sample (e.g. cells, microorganisms) on a substrate by an imaging device having a viewing zone, the substrate comprising an addressable array of encoded microstructures at a plurality of locations, each encoded microstructure being associated with an indexed position on the substrate, wherein the encoded microstructures are computer-readable to allow a controller or user to direct the imaging device to align at least a portion of the viewing zone to include a target location on the substrate based on the indexed position on the substrate.

### CROSS-REFERENCE TO RELATED APPLICATIONS

This application claims the benefit and priority to U.S. Provisional Application No. 63/638,153, filed Apr. 24, 2024, entitled “MICROCODES,” which is incorporated by reference herein in its entirety.

### STATEMENT OF GOVERNMENT SUPPORT

This invention was made with government support under Grant No. W 81XWH-20-1-0649 (A.F.S.) awarded by the Office of the Assistant Secretary of Defense for Health Affairs. The government has certain rights in the invention.

### BACKGROUND

Devices for imaging are useful in a wide range of research and industrial applications. For example, scanning electron microscopes use electrons to form images of extremely small features of samples. In a scanning electron microscope, a specimen is prepared and an electron beam is fired at the specimen, with backscattered electrons being detected and used to form an image of the specimen.

Optical or light microscopy involves passing visible light transmitted through or reflected from the sample through a single lens or multiple lenses to allow a magnified view of the sample. Common specimens can include biological samples and semiconductor devices. Other imaging devices include transmission electron microscopes and other high-resolution microscopy technologies, including those using visual light.

The ability to accurately map, track and discriminate individual cells/particles on a common substrate during provides important capabilities to researchers. However, current platforms generally rely on manual localization and lack robust spatial referencing systems, making it difficult to revisit a specific target by relocating the same field of view after changes to the equipment or between instruments.

Thus, there are benefits to improving the accuracy and usability of high-resolution imaging and also for spatial multiplexing of measurements by creating many uniquely identifiable spots on a substrate.

### SUMMARY

Systems, methods, and devices are disclosed that use micro and/or nano-sized unique spatial features or guide marks (also referred to as codes) physically formed or embedded into a workpiece or sampling structures (e.g., polymer-based workpiece or sampling structures, e.g., microscope slides). The codes can optionally be fabricated as alphanumerical text, represented in various coordinate systems and numeral systems, or designed as barcodes, QR codes, or other coding systems. Optionally the codes described herein can include alignment marks to aid in orientation of and/or compensate for errors in imaging. Coded symbol structures can be fabricated near the fabrication limits of fabrication processes, using existing capabilities of such processes, to provide finer control (more spatial resolution) and physical navigation facilitators or guide marks that are physically small so as to not affect the viewing or analysis of samples viewed on the workpiece/sampling structure. Preferably, the coded symbol structures are fabricated with a scaling to be in one field-of-view frame of the instrument of interest as such pattern would allow localization using one field of view.

The navigation facilitators or coded embedded in the workpiece or sampling structures can operate with microscopes or instruments that can use the physical structures for localization or navigation along the workpiece or sampling structures. Navigation operations are provided that allow (i) a first instrument to be employed for the viewing, inspection, and/or analysis of a workpiece/sampling structures or a sample on the workpiece/sampling structure and (ii) a second instrument, e.g., of greater or different viewing (e.g., field of view), inspection, or analysis capability (e.g., fluoroscopy, etc.) to be subsequently employed to performed additional analysis for the same sample or workpiece/sampling structure portion.

The coded symbol structures are preferably formed of native material of the workpiece or sampling structures and at the limits of the fabrication process for such workpiece or sampling structures, thus, can be made with de minimis incremental cost of the fabrication of the workpiece or sampling structures, though a different material and/or process (e.g., auxiliary process) may be employed, in alternative embodiments, to form the navigation facilitators or guide marks on the surface of the workpiece or sampling structures after the workpiece or sampling structures have been fabricated.

In various aspects, described herein is an apparatus comprising: a substrate for measuring, viewing, identifying, or inspecting a sample (e.g. cells, microorganisms) by an imaging device having a viewing zone, the substrate comprising an addressable array of encoded microstructures at a plurality of locations, each encoded microstructure being associated with an indexed position on the substrate, wherein the encoded microstructures are computer-readable to allow a controller or user to direct the imaging device to align at least a portion of the viewing zone to include a target location on the substrate based on the indexed position on the substrate.

In some aspects, the encoded microstructures are integrally formed with the substrate.

In some aspects, a size (e.g., a horizontal and/or vertical distance) of the viewing zone is greater than or equal to a distance between adjacent encoded microstructures (e.g., such that one or more encoded microstructures are visible within the viewing zone).

In some aspects, the encoded microstructures are fabricated as three-dimensional (3D) features on the substrate.

In some aspects, each of the encoded microstructures are formed to encode a distinctive barcode.

In some aspects, the distinctive barcode comprises a Postal Alpha Numeric Encoding Technique (PLANET) barcode.

In some aspects, the distinctive barcode comprises an alpha-numeric symbol.

In some aspects, the encoded microstructures of the addressable array are uniformly distributed about the substrate.

In some aspects, each of the encoded microstructures encodes a coordinate of the respective position on the substrate.

In some aspects, the substrate and/or the encoded microstructures comprise an optically transparent material.

In some aspects, the substrate comprises a well plate.

In some aspects, the substrate comprises a filter.

Also described herein are systems. In some aspects, the system includes: a computing device operably coupled to the imaging device, the imaging device being configured to receive the apparatus of claim 1; wherein the computing device is configured to: receive a signal of an image from the imaging device, wherein the image includes a representation of at least one encoded microstructure from the addressable array present in in the image; and determine a reference position of the at least one encoded microstructure present in the image relative to the substrate.

In some aspects, the imaging device comprises an optical microscope, a confocal microscope, or an electron microscope (e.g., SEM, TEM, CryoEM).

In some aspects, the computing device is further configured to output the determined reference position of the at least one encoded microstructure (e.g., to a memory unit).

In some aspects, the computing device is further configured to: calculate, based on a positional index (e.g., and a magnification of the imaging device), an adjustment factor to adjust the viewing zone from the reference position to the target position on the substrate.

In some aspects, the computing device is further configured to adjust the viewing zone (e.g., by moving the apparatus or by moving the viewing zone) based on the adjustment factor such that the viewing zone is aligned to include at least a portion of the target location.

In some aspects, the system further includes: a second imaging device having a second viewing zone, the second imaging device being configured to receive the apparatus of claim 1, wherein the system (e.g., via the computing device or a second computing device) is configured to: receive a signal of a second image from the second imaging device, wherein the second image includes a representation of at least one encoded microstructure from the addressable array present in in the second image; decode one or more of the at least one encoded microstructure(s) present in the second image to determine a starting point relative to the substrate; and adjust the second viewing zone of the second imaging device to include the reference position on the substrate based on the output and the determined starting point.

In some aspects, the computing device is configured to receive, via a graphical user interface, an input from a user associated with the target position on the substrate, and wherein the computing device is configured to determine an adjustment factor to adjust the viewing zone of the imaging device from the reference position to the target position.

In some aspects, the reference position and target position are the same.

Also described herein are method of operating an imaging device.

In some aspects, the method includes: receiving an image (e.g., of a scan or view) from an imaging device showing at least one encoded microstructure in an addressable array of encoded microstructures, each encoded microstructure being associated with an indexed position on a substrate; and determining a reference position by decoding the indexed position(s) of the at least one encoded microstructure present in the image relative to the substrate.

In some aspects, the method further includes: outputting the determined reference position (e.g., to a memory unit).

In some aspects, the method further includes: receiving a second image of the substrate from a second imaging device showing one or more of the at least one of the encoded microstructures in a second viewing zone; decoding the encoded microstructure(s) present in the second image to determine a starting point associated with a relative location on the substrate; and directing the second imaging device (e.g., based on an adjustment factor) to adjust the second viewing zone (e.g., by moving the substrate or by moving the second viewing zone) from the starting point to a target location on the substrate.

In some aspects, the method further includes: directing the imaging device (e.g., based on an adjustment factor) to adjust a viewing zone (e.g., by moving the substrate or by moving the viewing zone) of the of the imaging device from the reference position to a target location.

In some aspects, the target location is determined based on an input from a user (e.g., via a graphical user interface).

In some aspects, the target location is associated with an indexed position associated with a location of a sample (e.g. cells, microorganisms) on the substrate.

In some aspects, the method further includes: receiving a second image of at least one of the encoded microstructures from the imaging device, and, based on the encoding formed in the encoded microstructures, identifying a movement of a sample disposed on the substrate.

Advantageously, aspects of the present disclosure can be used to navigate samples with small field of view (FOV) relative to the total size of a sample. An example application can include a small FOV is scanning electron microscopy or an optical microscope. In an image with a very small field of view, a marker can be useful to identify where on a sample the image was captured. Embodiments of the present disclosure can therefore be used to combine data from across multiple images captured by different instruments (e.g., confocal microscopes, electron microscopes, and/or optical microscopes), for example by using one or more markers to establish a common reference point across the images.

As yet another application, embodiments of the present disclosure can be used to mark a sample of captured cancer cells and track them from isolation to characterization and analysis.

Additionally, embodiments of the present disclosure include methods that can be used to convert the location of encoded microstructures in an image to microscope coordinates (e.g., the 3D position of the microcode relative to a known point in space), allowing for samples to be tracked in 3D.

An example embodiment of the present disclosure includes an optical transparent structure for measuring, viewing, or inspecting a sample. Optionally the sample can be cells or other organisms. The optical transparent structure can be configured for viewing with an imaging device having a viewable zone. The optical transparent substrate can be fabricated with features encoded that are computer-readable to allow a controller or user to direct the imaging device to position the viewable zone to an encoded position.

An example embodiment of the present disclosure includes a method of operating imaging device. The example method can include receiving an image of an optical transparent structure. The image can optionally include a scan or view (e.g., scanning electron microscope scan, optical image view). The image can be configured for placement of a sample. The method can further include determining an encoding formed in the optical transparent structure, the optical transparent substrate having a plurality of encoded fabricated into the structure at a plurality of locations to identify a location on the structure. The method can further include receiving, via a graphical user interface, an input from a user for an encoding or a location; directing the imaging device to move the optical transparent structure or a viewable zone of the optical transparent structure to a location associated with the input.

In yet another example embodiment can include a system including transparent optical structures and an imaging device. The imaging device can include optionally be an optical microscope, electron microscope, and/or any other type of microscope. The system can further include a plurality of optical transparent structures, where the optical transparent structures each comprising an encoding feature formed in the transparent optical structure.

A computing device can be operably coupled to the imaging device, where the computing device can be configured to perform any of the methods described herein. In some embodiments, the computing device can receive an image from the imaging device, wherein the image includes at least one transparent optical structure from the plurality of optical transparent structures present in in the image; and output a position of the at least one transparent optical structure present in the image.

Embodiments of the present disclosure further include methods of fabricating transparent imaging devices.

### BRIEF DESCRIPTION OF DRAWINGS

The skilled person in the art will understand that the drawings described below are for illustration purposes only.

FIGS. 1A-1C each show an exemplary system for facilitating navigation and alignment in order to measure, view, identify, or inspect a sample on a substrate according to one aspect of the present disclosure.

FIG. 2 shows an example method for operating an imaging device according to one aspect of the present disclosure.

FIG. 3 shows an example of binary microcode fabricated into a cluster wells filter.

FIG. 4 shows an example of scanning a filter, finding a cell of interest and locating the nearby microcode.

FIG. 5 shows an example equation for determining displacement between a reference and a target microcode.

FIG. 6 shows an example of decimal numbers creating a guideline grid.

FIG. 7 shows a screenshot of a software application configured to predict the location of the object of interest.

FIG. 8 shows an example of navigation and location with microcodes shows use of microcodes to locate target microcodes in the optical and electron microscope.

FIG. 9 shows an example of pipeline of processes that microcodes can be used in to assist the user.

FIG. 10 shows (Panel a) an exemplary fabrication technique for forming (Panel b) an image of a fabricated silicon mold (Panel c) a fabricated filter having encoded microstructures.

FIG. 11 shows example of fabricated filter showcasing the codes.

FIG. 12 shows examples of fabricated codes.

FIG. 13 shows example of fabricated microcodes as QR codes.

FIG. 14 shows example of fabricated microcodes as POSNET/PLA NET codes.

FIG. 15 shows example of fabricated microcodes in base 3.

FIG. 16 shows example of fabricated microcodes in base 2.

FIG. 17 shows example of fabricated microcodes in decimal.

FIG. 18 shows example of special binary microcodes with Cartesian coordinates, designed to fit available spaces.

FIG. 19 shows example of special binary microcodes with Cartesian coordinates, designed to fit available spaces.

FIG. 20 shows example of minimally observed codes, designed to have minimal impact of the main structure.

FIG. 21 shows example HEY A8 cells shows locating the same cell in fluorescent (left) and scanning electron microscope (right).

FIG. 22 shows an exemplary computing device.

### DETAILED DESCRIPTION

### General Definitions

Unless defined otherwise, all technical and scientific terms used herein have the same meaning as commonly understood by one of ordinary skill in the art. Methods and materials similar or equivalent to those described herein can be used in the practice or testing of the present disclosure.

As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not.

Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. W hile implementations will be described for measuring soil, it will become evident to those skilled in the art that the implementations are not limited thereto, but are applicable for any other type of sensing.

As used herein, by a “subject” is meant an individual. Thus, the “subject” can include domesticated animals (e.g., cats, dogs, etc.), livestock (e.g., cattle, horses, pigs, chickens, ducks, geese, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

“Detecting” is used herein to identify the existence, presence, or fact of something. General methods of detecting are known to the skilled artisan and may be supplemented with the protocols and reagents disclosed herein. For example, included herein are methods of detecting a nucleic acid molecule in sample. Detection can include a physical readout, such as fluorescence output.

An “isolated” biological component (such as a nucleic acid molecule) has been substantially separated, produced apart from, or purified away from other biological components. Nucleic acid molecules which have been “isolated” include nucleic acids molecules purified by standard purification methods, as well as those chemically synthesized. Isolated does not require absolute purity, and can include nucleic acid molecules that are at least 50% isolated, such as at least 75%, 80%, 90%, 95%, 98%, 99% or even 100% isolated.

A “sample,” such as a biological sample, is a sample obtained from a subject. As used herein, biological samples include all clinical samples, including, but not limited to, cells, tissues, and bodily fluids, such as: blood; derivatives and fractions of blood, such as serum; urine; sputum; or CVS samples. In a particular example, a sample includes blood obtained from a human subject, such as whole blood or serum. As used herein, the term “whole blood” refers to blood comprising blood plasma, which is typically unclotted, and cellular components. The plasma represents about 50 to about 60% of the volume, and cellular components, i.e. erythrocytes (red blood cells, or RBCs), leucocytes (white blood cells, or W BCs), and thrombocytes (platelets), represent about 40 to about 50% of the volume. Reference to “whole blood” may refer to whole blood of an animal, such as whole blood of a human subject.

As used herein, the term “array” refers to an ordered spatial arrangement, particularly an arrangement of features (e.g., encoded microstructures) on a substrate. Similarly, the term “addressable array” refers to an array wherein the individual elements have known or determinable coordinates, so that a given element at a particular position in the array can be identified.

The term “distinctive,” or “unique,” as used herein, generally refers to an object that is distinguishable from other objects in a group. The term “encoded microstructure” can refer to a distinctive feature on a substrate that stores or preserves spatial information (e.g., a particular location on the substrate) by way of its shape. Encoded microstructures can include both features representing numerical/textual values as well as unique shapes that do not contain a particular semantic meaning (e.g., pixels or scratches).

As used herein, the term “imaging device” can refer to any number or combination of devices and associated computer hardware and software that can be configured to collect or generate images of a target, such as the substrate and the encoded microstructures as described herein. Non-limiting examples of imaging devices can include: scanning electron microscopes (SEMs), atomic force microscopes (AFMs), x-ray machines, optical microscopes, etc.

### Example Systems and Apparatuses

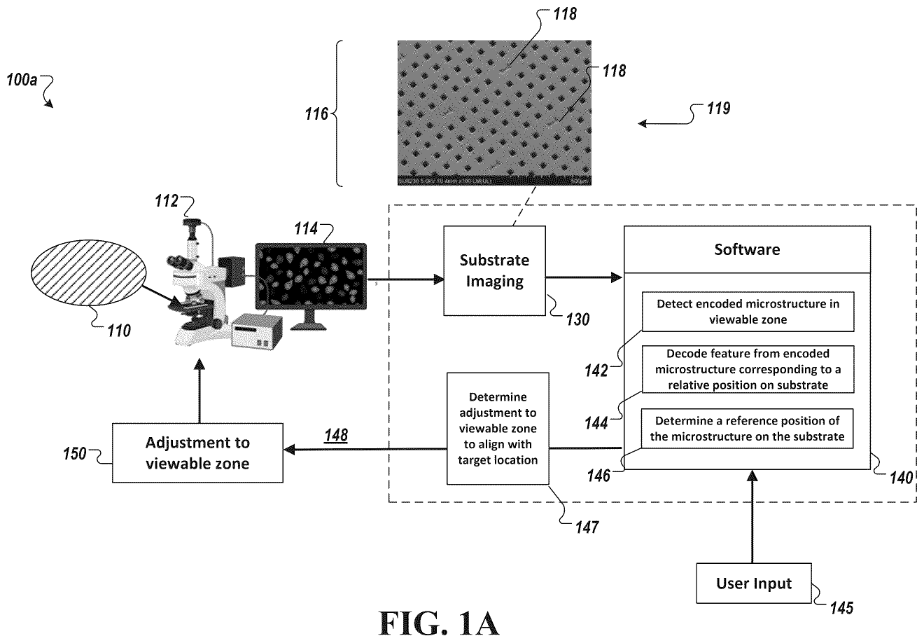

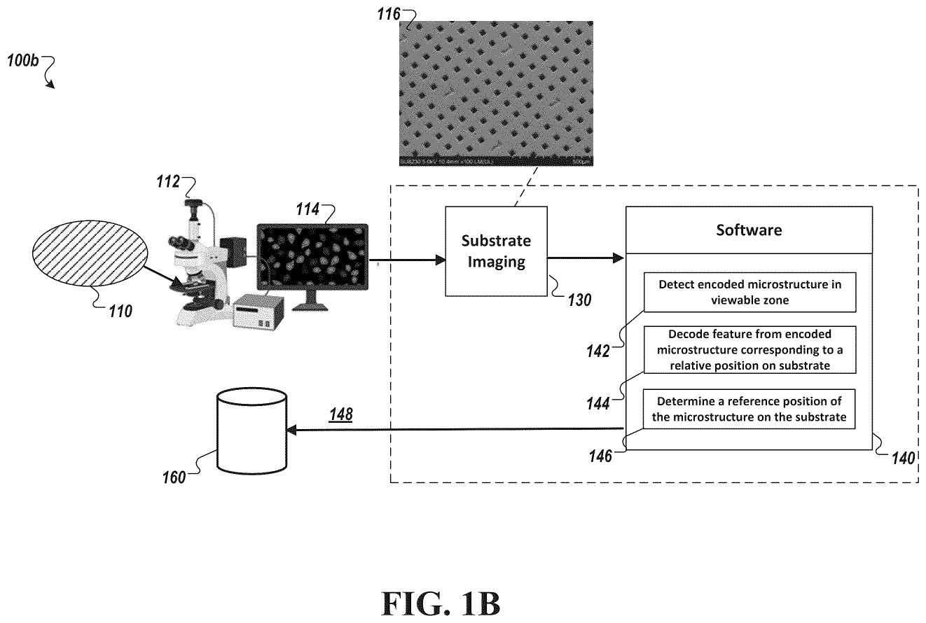

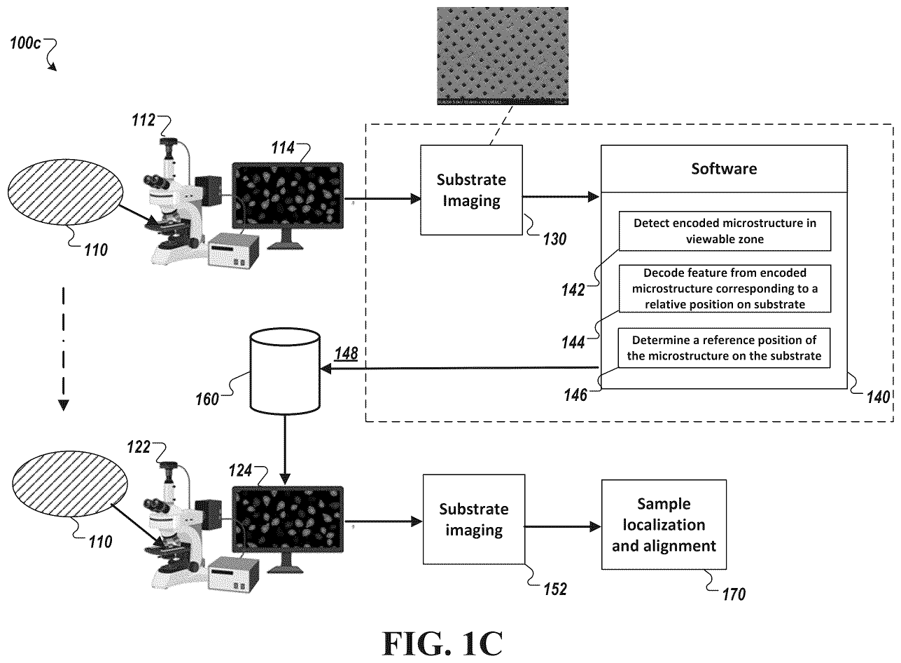

FIGS. 1A, 1B, and 1C each illustrate exemplary systems 100 (shown as 100a, 100b, 100c respectively) for facilitating navigation and alignment in order to measure, view, identify, or inspect a sample on a substrate 116 according to one aspect of the present disclosure.

The system 100 includes an imaging device 112, and a computing device 114 operably coupled to the imaging device 112. The imaging device 112 is configured to receive an apparatus 110 including a substrate 116 comprising an addressable array of encoded microstructures 118 (also referred to as “microcodes”) at a plurality of locations on a surface of the substrate 116. Each encoded microstructure 118 is associated with an indexed position on the substrate 116. As used herein, the term “indexed position” refers to a designated location of a feature within an array of features which is fixed relative to other features in the array to provide a relative or absolute positional value with respect to the substrate.

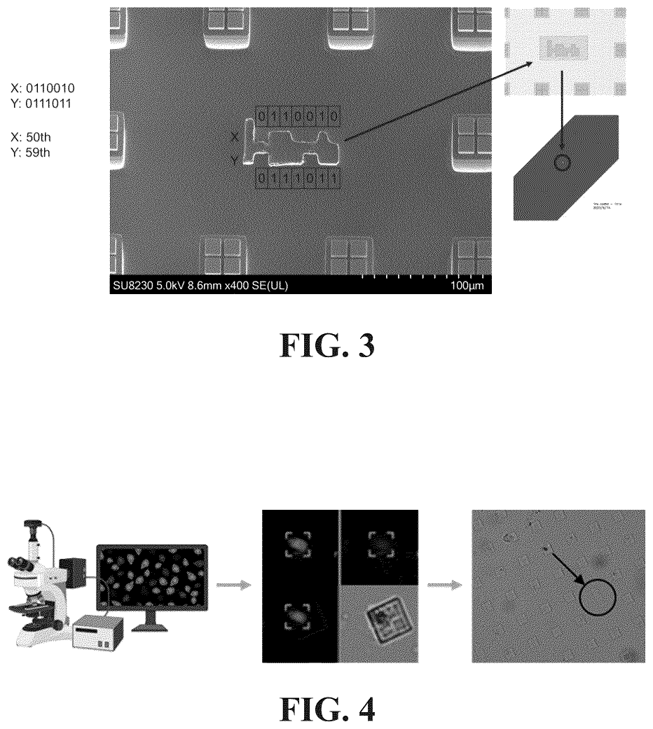

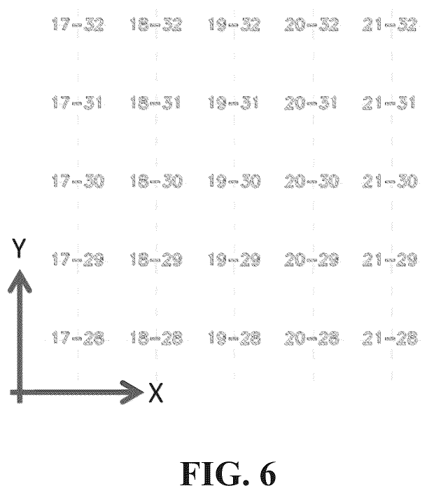

The encoded microstructures 118 are shaped as computer-readable identifiers to allow a controller (e.g., computing device 114) or a user to direct the imaging device 112 to align a viewing zone to include a target location on the substrate based on the indexed position on the substrate such as that of FIG. 6. As seen in FIG. 3, the encoded microstructure can be shaped to correspond to a set of binary values designating a coordinate on the substrate. Other examples of computer-readable identifier types include barcodes (e.g., Postal Alpha Numeric Encoding Technique (PLANET) barcode), as well as character sets of numbers, symbols, alphabetical letters, and/or spaces. In some aspects, the encoded microstructures are fabricated as three-dimensional (3D) features on the substrate.

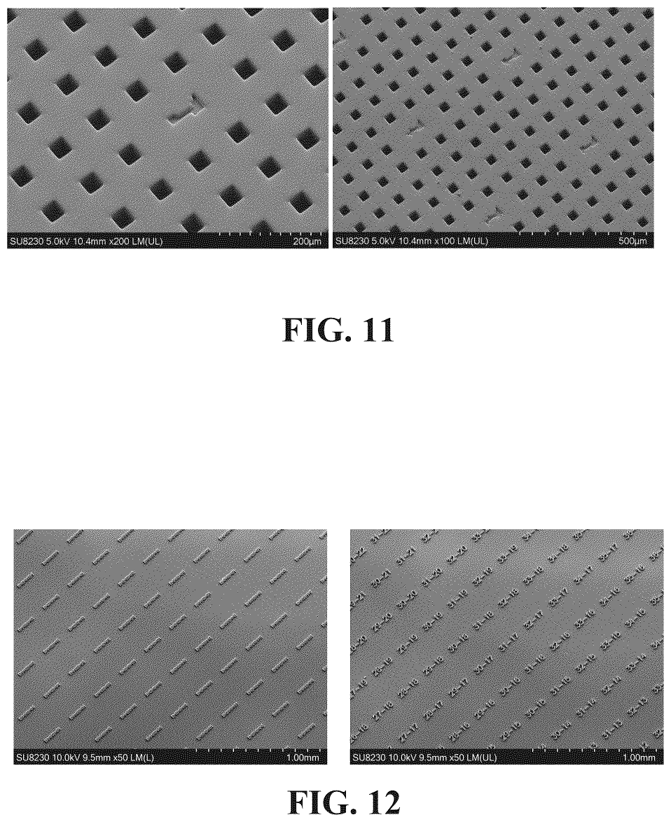

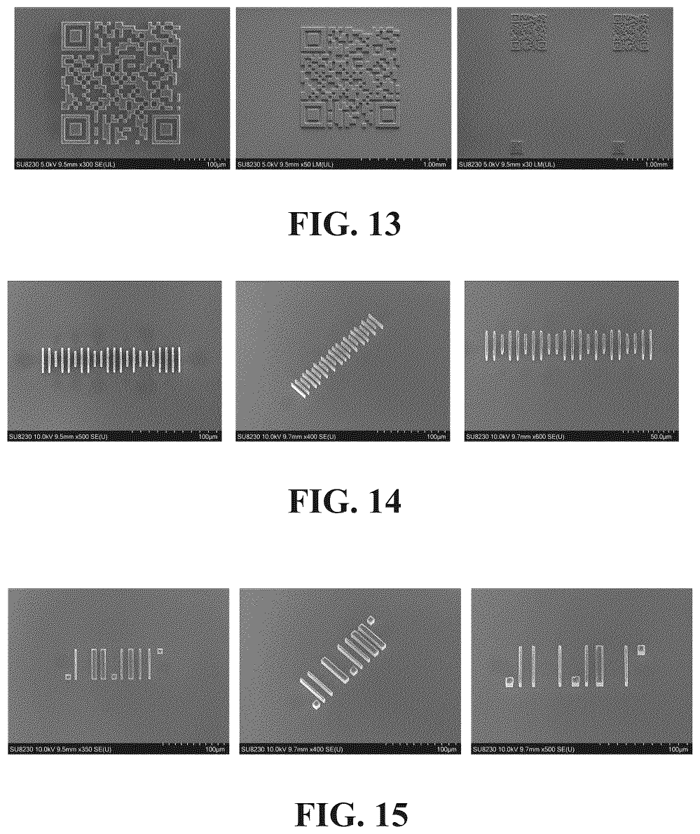

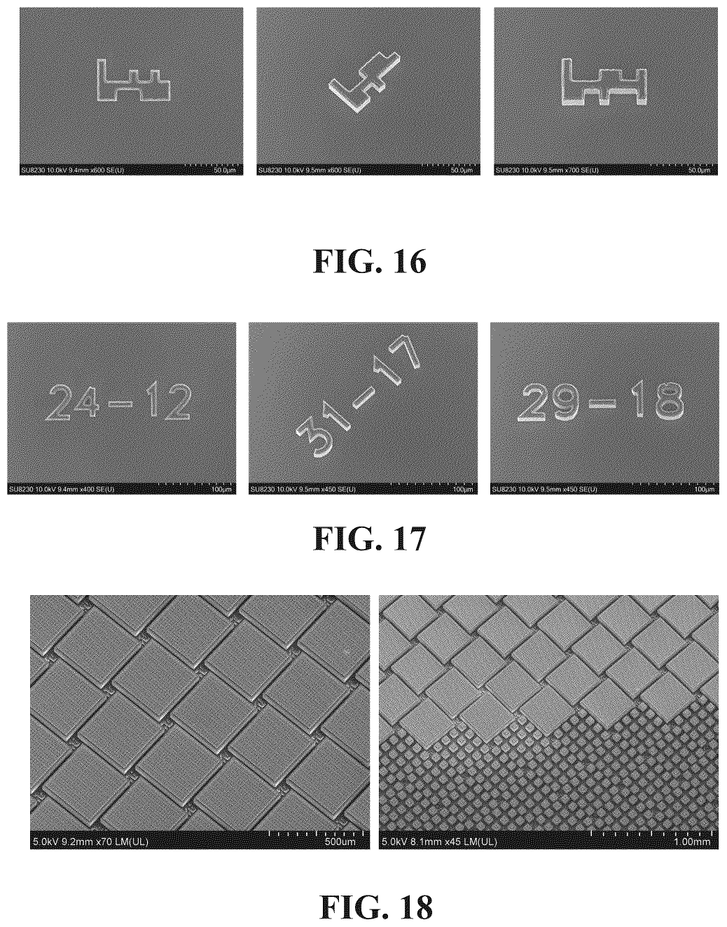

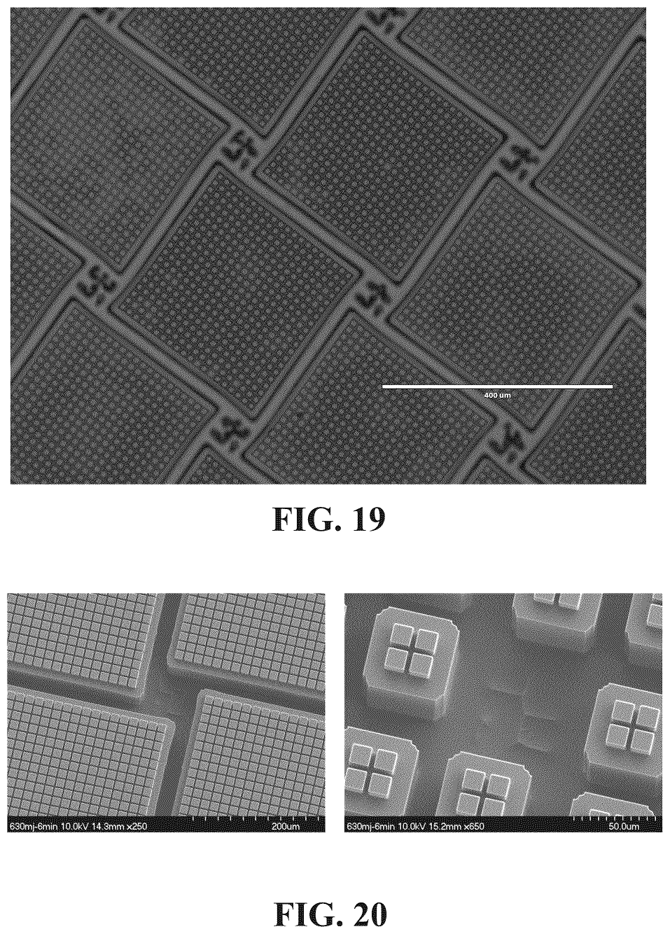

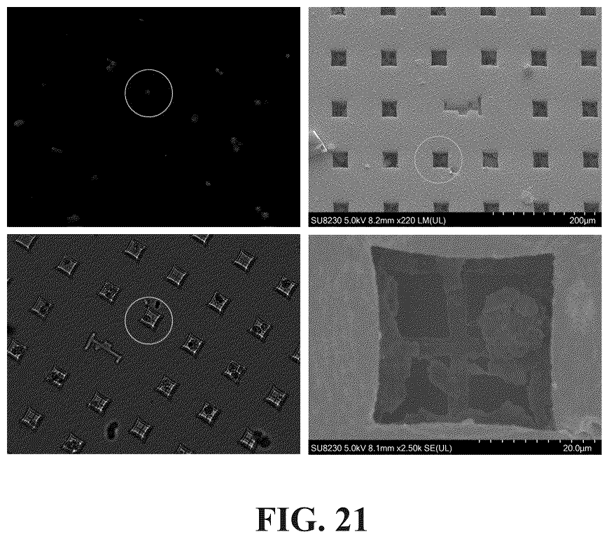

In some aspects, each of the encoded microstructures are formed to encode a distinctive barcode. In some aspects, the distinctive barcode comprises a Postal Alpha Numeric Encoding Technique (PLANET) barcode. In some aspects, the distinctive barcode comprises an alpha-numeric symbol. Several examples of these structures are shown in FIGS. 11-21. In some aspects, the encoded microstructures of the addressable array are uniformly distributed about the substrate. In other words, the addressable array of encoded microstructures is arranged in a pattern on the surface of the substrate such that distances between adjacent microstructures is substantially the same. The encoded microstructures can further be shaped such that an orientation of the substrate can be determined from the image.

In some aspects, the encoded microstructures are integrally formed with the substrate. A s used herein, the term “integrally formed” means the encoded microstructures are embedded or otherwise directly formed into surface of the substrate as a single continuous piece. Generally, a density of the encoded microstructures is sufficiently high such that at least one of microstructure will appear in the viewing zone of the imaging device. In some aspects, a size (e.g., a horizontal and/or vertical distance) of the viewing zone is greater than or equal to a distance between adjacent encoded microstructures such that one or more encoded microstructures are visible within the viewing zone. In some aspects, a surface density of the encoded microstructures on the substrate is 500 encoded microstructures/mm² or more (1000 microstructures/mm² or more, 1500 microstructures/mm² or more, 2000 microstructures/mm² or more, 2500 microstructures/mm² or more, or 3000 microstructures/mm² or more). In some aspects, each of the microstructures has a size of from 1 μm to 1000 μm (e.g., from 1 μm to 900 μm, from 1 μm to 800 μm, from 1 μm to 700 μm, from 1 μm to 600 μm, from 1 μm to 500 μm, from 1 μm to 400 μm, from 1 μm to 300 μm, from 1 μm to 200 μm, from 1 μm to 100 μm, from 1 μm to 50 μm, from 5 μm to 40 μm, from 5 μm to 30 μm, or from 5 μm to 20 μm).

In some aspects, each of the encoded microstructures encodes a coordinate of the respective position on the substrate. Depending on the particular application, the substrate and/or the encoded microstructures can be formed using various types of materials. In some aspects, the substrate and/or encoded microstructures comprise a polymer. N on-limiting examples of suitable polymers include, for example, polyethylene, low-density polyethylene, polymethylmethacrylate (PMMA), polyetheretherketone (PEEK), polyetherketoneketone (PEKK), and polyurethane. In some aspects, the substrate and/or the encoded microstructures comprise an optically transparent material. Optically transparent materials may be particularly advantageous for allowing use in certain imaging devices. Exemplary types of optically transparent materials include glass, plastic films including polyvinyl chloride, polyester, polyethylene, polypropylene and polycarbonate films.

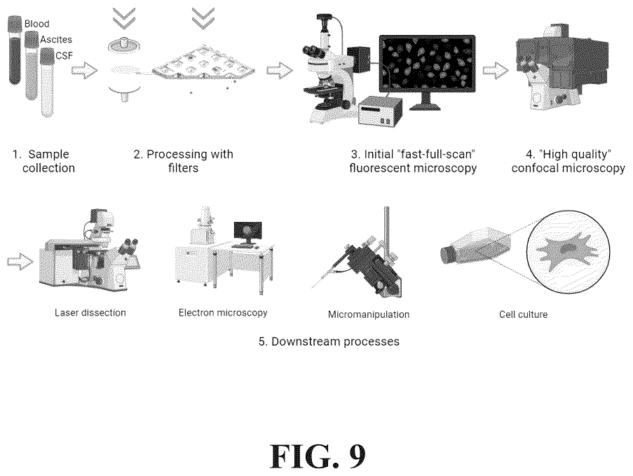

In some aspects, the apparatus comprises a well plate. In some aspects, the apparatus comprises a filter substrate. For example, the encoded microstructures can be formed on a filter substrate or a microscopy grid, such as those described WO2024/124055A1, which is expressly incorporated by reference herein in its entirety. FIG. 9 describes an exemplary use case for processing a sample with filters. Samples, including various biological fluids (e.g., blood, ascites, cerebral spinal fluid), may be processed through a microfiltration system designed to capture particles or cells of interest, optically mixed with a fluorophore or another type of marker. Next, the filter substrate can be imaged using a first imaging device (e.g., a fluorescent microscope) to quickly locate retained particles/cells. Following the procedures described herein, the positions of relevant particles/cells on the substrate can be labeled—which can subsequently be used to relocate the same particles/cells using another imaging device (e.g., confocal microscope). Additional downstream processing of individual particles/cells may also be performed to assist with diagnosis of a condition, research, or other clinical applications.

Substrate imaging 130 by the computing device 114 includes receiving and/or capturing an image 119 from the imaging device 112, wherein the image 119 includes at least one encoded microstructure 118 from the addressable array present in in the image 119. The image 119 may optionally include a step of image processing to obtain higher resolution images and/or to otherwise enhance detection of features (e.g., encoded microstructures) on the substrate 116. Numerous techniques for image processing are generally known in the art. By way of non-limiting example, these methods can include, but are not limited to, spatial rearrangement (e.g., rotation, translation, transformation), contrast enhancement, image sharpening, morphing, noise removal, edge detection, color enhancement and pedestal processing.

The computing device 114 in system 100 is configured, via software 140, to: detect (142) encoded microstructure present 118 in the viewable zone, decode (144) features from the encoded microstructures 118 associated with an indexed position on the substrate 116. Once decoded, the computing device can identify the unique microstructure and determine a reference position (146) on the substrate.

Referring specifically to FIG. 1A, the software 140 can be configured to receive input 145 from a user corresponding to a target position on the substrate. The target position can be associated with an indexed position of an encoded microstructure on the substrate corresponding to a point of interest (e.g., a location of a cell). Based on this indexed position, the software 140 is configured to determine an adjustment to the viewable zone to align with a target location 147. This determined adjustment 147 is then digitally transmitted as an output 148 to a data store 160 (such as those shown in FIGS. 1B and 1C) or to a controller or user to make an adjustment 150 to the viewable zone of the imaging device 112 as illustrated in FIG. 1A.

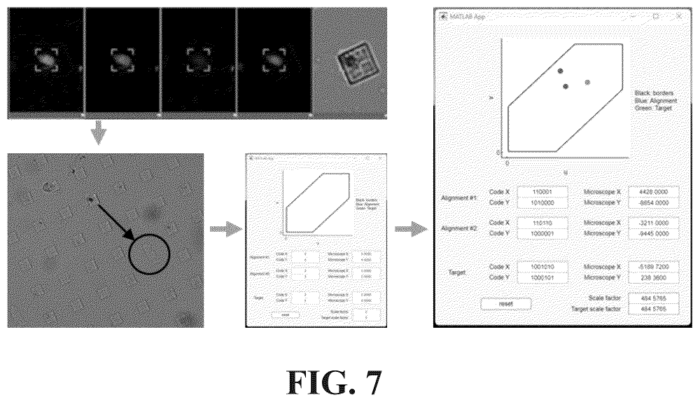

Detection (142) implemented by the software 140 can include scanning the image 119 (before or after optional image processing) to locate a computer recognizable feature associated with the addressable array of encoded microstructures 118. Exemplary detection schemes are shown in FIGS. 4 and 7, which depicts the localization of a sample under different fluorescent conditions. It is also advantageous in some cases to use encoded microstructures having alignment features denoting a common orientation of the microstructures on the substrate. In this regard, the computing device can effectively standardize detection of the features in the viewing zone regardless of the placement of the substrate within the imaging device.

The computing device 114 is further configured to determine (146) a reference position of the at least one encoded microstructure present in the image relative to the substrate 116. Determining (147) the adjustment to the to the view can include a comparison of the coordinates of the reference position and the target position. For example, a relative location of the reference position and target position can be determined by comparing their encoded values (e.g., the indexed position) to a lookup table and calculating a displacement (e.g., angle and/or distance) needed to align the viewing zone from the reference position to the target position. The term “lookup table” (or LUT) as used herein can refer to a data array that may include predetermined or reference data (e.g., coordinates of unique microstructures on the substrate) useable for comparison. A LUT(s) can be stored in static program storage, including solid data storage. In some aspects, the displacement can be scaled based on a difference in magnification between multiple imaging devices.

Referring now to FIG. 1B and FIG. 1C, the output 148 of the software 140 can be transmitted to a data store 160 for downstream imaging applications. The data store can include tangible, computer-readable recording media including, but not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, or magnetic storage devices as well as remote computer storage media. In various aspects, the data store can be co-located with the computing device.

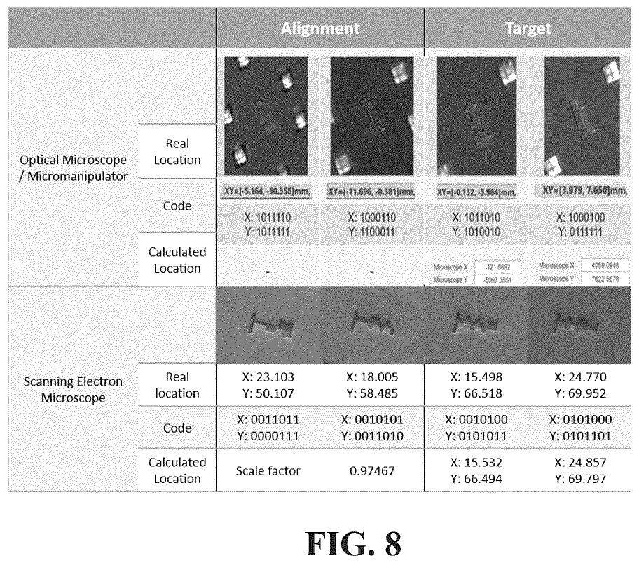

Referring specifically to FIG. 1C, the output 148 on the data store 160 can be transmitted to a second imaging device 122 having a second viewing zone. The second imaging device 122 is configured to receive the same apparatus 110 that was observed by the first imaging device 112 and record a second image. The system 100, via a second computing device 124 is configured to receive the signal of a second image from the second imaging device 122. Similar to the imaging via the first imaging device 112, the second image includes a representation of at least one encoded microstructure 118 from the addressable array present in the second image. Generally, the at least one encoded microstructure present in the second viewing zone during initialization is not the same as the encoded microstructure depicted in the first image. The second computing device 124 is further configured for substrate imaging via the second imaging device and for sample localization and/or alignment 170. The computing device is thus configured to decode one or more of the at least one encoded microstructure(s) present in the second image to determine a starting point relative to the substrate; and adjust the second viewing zone of the second imaging device to include the reference position on the substrate based on the output and the determined starting point. Although FIG. 1C shows two separate computing devices (114, 124), it is also envisioned that the respective processing for systems with multiple imaging devices may occur on a single computing device. Furthermore, in other aspects, the system may include a plurality (e.g., 2 or more, 3 or more, 4 or more, 5 or more, etc.) of imaging devices and/or computing devices. FIG. 8 illustrates how encoded microstructures can be observed using different imaging devices.

The system 100 may also include a display (which may be co-located with the computing device 114) for visually presenting information associated with the described methods. The display may comprise, for example, a computer monitor (e.g., LCD, a CRT monitor, a projection (e.g., heads-up display (HUD) laser), etc. In some embodiments, the visual display may comprise, for example, that of a mobile device such as a tablet computer, cellular phone, smartphone, personal digital assistant (PDA), personal computer (PC), laptop computer, augmented reality display (e.g., Google™ Glass™ or Microsoft™ HoloLens™), etc. The information presented on the display may include measurements obtained by or derived from the sensor data, for example, and/or may include any other information collected in the course of carrying out the methods described herein, prompts for information entry associated with one or more steps of the described methods, and/or any predetermined formulae or algorithms, as previously described. The display may also be capable of receiving input (such as, e.g., where the display includes a touch-screen and is capable of receiving touch input and accordingly transmitting information to the computing device 114).

As the simplest design, microstructures can be regular numbers fabricated into the structure. Microcodes can further designate an x and an y coordinate or be made in any coordinate system such as Cartesian, Polar, Cylindrical, and spherical, etc. In some aspects, the encoded microstructures include an alignment mark. Alignment marks in the encoded microstructure can help compensate for orientation, flipping and/or angular errors.

### Example Methods

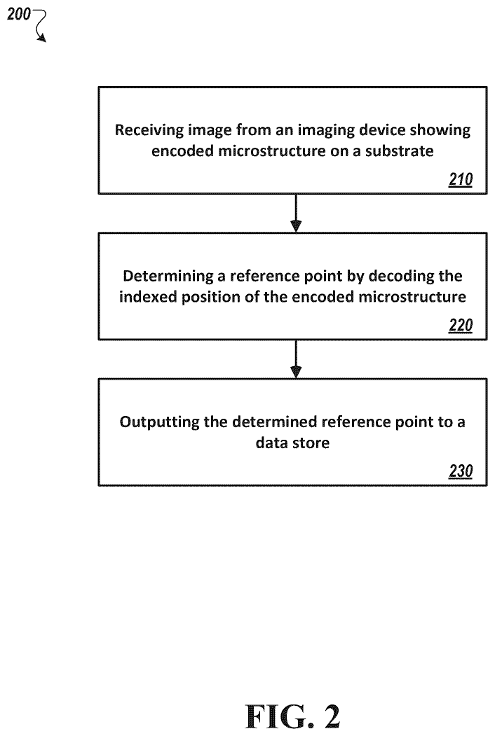

FIG. 2 illustrates an example method 200 of operating an imaging device.

Method 200 includes: receiving 210 an image (e.g., of a scan or view) from an imaging device showing at least one encoded microstructure in an addressable array of encoded microstructures. Each encoded microstructure is associated with an indexed position on a substrate. Method 200 further includes determining (220) a reference position by decoding the indexed position(s) of the at least one encoded microstructure present in the image relative to the substrate.

The reference position can then be outputted (230) to a data store for subsequent localization and/or tracking of the sample.

In some aspects, the method further includes: receiving a second image of the substrate from a second imaging device showing one or more of the at least one of the encoded microstructures in a second viewing zone; decoding the encoded microstructure(s) present in the second image to determine a starting point associated with a relative location on the substrate; and directing the second imaging device (e.g., based on an adjustment factor) to adjust the second viewing zone (e.g., by moving the substrate or by moving the second viewing zone) from the starting point to a target location on the substrate.

In some aspects, the method further includes: directing the imaging device (e.g., based on an adjustment factor) to adjust a viewing zone (e.g., by moving the substrate or by moving the viewing zone) of the of the imaging device from the reference position to a target location.

In some aspects, the target location is determined based on an input from a user (e.g., via a graphical user interface).

In some aspects, the target location is associated with an indexed position associated with a location of a sample (e.g. cells, microorganisms) on the substrate.

In some aspects, the method further includes: receiving a second image of at least one of the encoded microstructures from the imaging device, and, based on the encoding formed in the encoded microstructures, identifying a movement of a sample disposed on the substrate.

### Example Case Study

An example embodiment can be used in conjunction with filters for isolation of cancer cells from blood. Microstructures can follow the captured cells through their process of being captured, characterized, and analyzed. After the cells are captured, the filter is scanned with a microscope of lower quality. Then the high value cells and the closest encoded microstructure to the target cells is identified. Then based on the downstream analysis, the encoded microstructures are used for locating the cells for further high-quality imaging, micromanipulation, or laser dissection for following or further genetical analysis, etc.

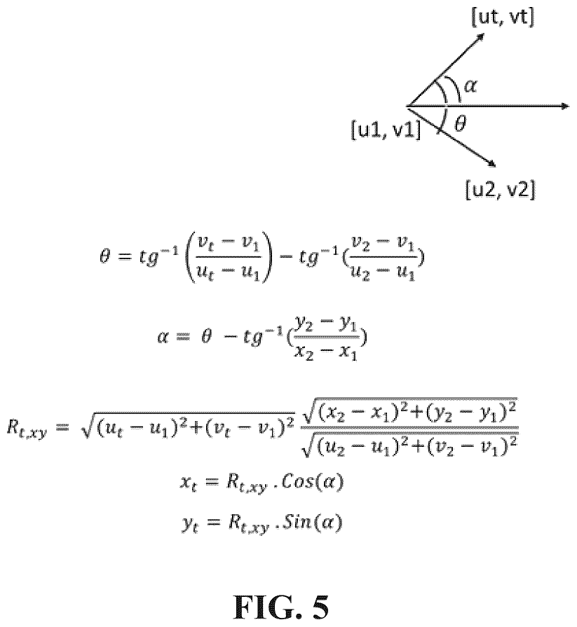

An example MATLAB application was written including a method to convert location from encoded microstructures to microscope coordinates. The exemplary method includes two steps: two random encoded microstructures and their locations are entered to the program. After identifying the target of interest and the nearby encoded microstructure, the encoded microstructure of interest is given to the program. Then, based on the known locations of the first encoded microstructures, the program calculates the location of the target microstructure, e.g., per the equation shown in FIG. 5.

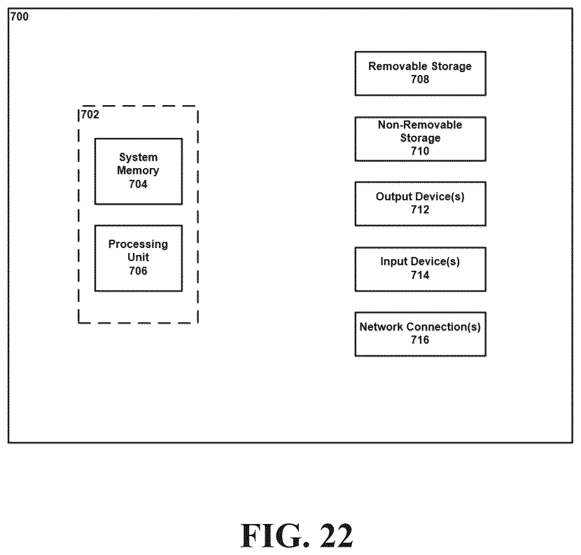

It should be appreciated that the logical operations described herein with respect to the various figures may be implemented (1) as a sequence of computer implemented acts or program modules (i.e., software) running on a computing device (e.g., the computing device described in FIG. 22), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 22, an example computing device 700 upon which the methods described herein may be implemented is illustrated. It should be understood that the example computing device 700 is only one example of a suitable computing environment upon which the methods described herein may be implemented. Optionally, the computing device 700 can be a handheld or laptop devices, multiprocessor systems, microprocessor-based systems, network personal computers (PCs), minicomputers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 700 typically includes at least one processing unit 706 and system memory 704. Depending on the exact configuration and type of computing device, system memory 704 may be volatile (such as random access memory (RAM)), non-volatile (such as read-only memory (ROM), flash memory, etc.), or some combination of the two. This most basic configuration is illustrated in FIG. 17 by dashed line 702. The processing unit 706 may be a standard programmable processor that performs arithmetic and logic operations necessary for operation of the computing device 700. The computing device 700 may also include a bus or other communication mechanism for communicating information among various components of the computing device 700.

Computing device 700 may have additional features/functionality. For example, computing device 700 may include additional storage such as removable storage 708 and non-removable storage 710 including, but not limited to, magnetic or optical disks or tapes. Computing device 700 may also contain network connection(s) 716 that allow the device to communicate with other devices. Computing device 700 may also have input device(s) 714 such as a keyboard, mouse, touch screen, etc. Output device(s) 712 such as a display, speakers, printer, etc. may also be included. The additional devices may be connected to the bus in order to facilitate communication of data among the components of the computing device 700. All these devices are well known in the art and need not be discussed at length here.

The processing unit 706 may be configured to execute program code encoded in tangible, computer-readable media. Tangible, computer-readable media refers to any media that is capable of providing data that causes the computing device 700 (i.e., a machine) to operate in a particular fashion. Various computer-readable media may be utilized to provide instructions to the processing unit 706 for execution. Example tangible, computer-readable media may include, but is not limited to, volatile media, non-volatile media, removable media and non-removable media implemented in any method or technology for storage of information such as computer readable instructions, data structures, program modules or other data. System memory 704, removable storage 708, and non-removable storage 710 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, or magnetic storage devices.

In an example implementation, the processing unit 706 may execute program code stored in the system memory 704. For example, the bus may carry data to the system memory 704, from which the processing unit 706 receives and executes instructions. The data received by the system memory 704 may optionally be stored on the removable storage 708 or the non-removable storage 710 before or after execution by the processing unit 706.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, hard drives, or any other machine-readable storage medium where, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### Fabrication Methods

As an example, methods of forming the above substrate can include fabricating a negative pattern mold of a substrate having the encoded microstructures on a negative pattern glass substrate as a build plate. Briefly, a negative structure with the encoded microstructures can be microfabricated into a master mold (e.g., PET sheet, patterned glass, or metal) using a variety of techniques known in the art, including but not limited to photolithography, soft lithography, hot embossing, laser ablation, wet etching, plasma etching (e.g. reactive ion etching or deep reactive ion etching), and/or micromolding. After the negative structure has been microfabricated into master mold, a first polymer sheet can be contacted to the master mold to form a reverse of the negative structure. Then, a second polymer sheet can be contacted with the first polymer sheet to form the negative pattern mold of the substrate with the microstructures.

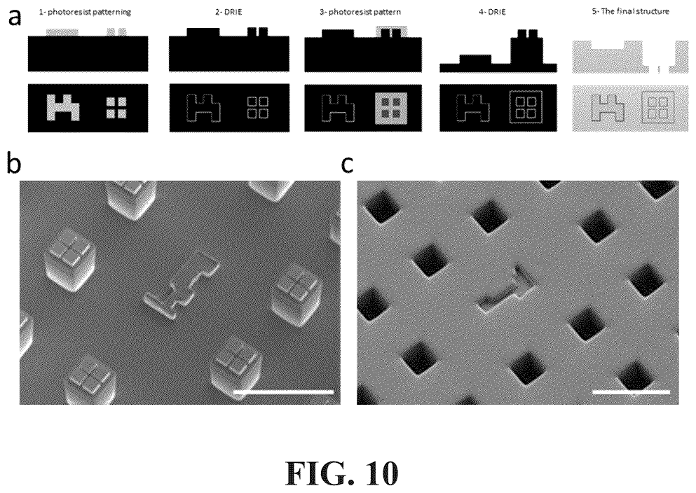

An example fabrication method includes transferring features into the structure as shown in FIG. 10. In some use cases such as the cell isolating filters, the features can be formed on a surface of a part such as a well plate, or a filter substrate, or a microscopy grid.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. An apparatus comprising:
a substrate for measuring, viewing, identifying, or inspecting a sample by an imaging device having a viewing zone, the substrate comprising an addressable array of encoded microstructures at a plurality of locations, each encoded microstructure being associated with an indexed position on the substrate, wherein the encoded microstructures are computer-readable to allow a controller or a user to direct the imaging device to align at least a portion of the viewing zone to include a target location on the substrate based on the indexed position on the substrate.

2. (canceled)

3. The apparatus of claim 1, wherein a size of the viewing zone is greater than or equal to a distance between adjacent encoded microstructures.

4. (canceled)

5. The apparatus of claim 1, wherein each of the encoded microstructures are formed to encode a distinctive barcode.

6. The apparatus of claim 5, wherein the distinctive barcode comprises a Postal Alpha Numeric Encoding Technique (PLANET) barcode.

7. The apparatus of claim 5, wherein the distinctive barcode comprises an alpha-numeric symbol.

8. (canceled)

9. (canceled)

10. (canceled)

11. (canceled)

12. (canceled)

13. A system comprising:
a computing device operably coupled to the imaging device, the imaging device being configured to receive the apparatus of claim 1; wherein the computing device is configured to:
receive a signal of an image from the imaging device, wherein the image includes a representation of at least one encoded microstructure from the addressable array present in in the image; and
determine a reference position of the at least one encoded microstructure present in the image relative to the substrate.

14. The system of claim 13, wherein the imaging device comprises an optical microscope, a confocal microscope, or an electron microscope.

15. The system of claim 13, wherein the computing device is further configured to output the determined reference position of the at least one encoded microstructure.

16. The system of claim 13, wherein the computing device is further configured to:
calculate, based on a positional index, an adjustment factor to adjust the viewing zone from the reference position to the target position on the substrate.

17. The system of claim 16, wherein the computing device is further configured to adjust the viewing zone based on the adjustment factor such that the viewing zone is aligned to include at least a portion of the target location.

18. The system of claim 15, further comprising:
a second imaging device having a second viewing zone, the second imaging device being configured to receive the apparatus of claim 1, wherein the system is configured to:
receive a signal of a second image from the second imaging device, wherein the second image includes a representation of at least one encoded microstructure from the addressable array present in in the second image;
decode one or more of the at least one encoded microstructure(s) present in the second image to determine a starting point relative to the substrate; and
adjust the second viewing zone of the second imaging device to include the reference position on the substrate based on the output and the determined starting point.

19. The system of claim 13, wherein the computing device is configured to receive, via a graphical user interface, an input from a user associated with the target position on the substrate, and wherein the computing device is configured to determine an adjustment factor to adjust the viewing zone of the imaging device from the reference position to the target position.

20. The system of claim 13, wherein the reference position and target position are the same.

21. A method of operating an imaging device comprising:
receiving an image from an imaging device showing at least one encoded microstructure in an addressable array of encoded microstructures, each encoded microstructure being associated with an indexed position on a substrate; and
determining a reference position by decoding the indexed position(s) of the at least one encoded microstructure present in the image relative to the substrate.

22. The method of claim 21, further comprising: outputting the determined reference position.

23. The method of claim 22, further comprising:
receiving a second image of the substrate from a second imaging device showing one or more of the at least one of the encoded microstructures in a second viewing zone;
decoding the encoded microstructure(s) present in the second image to determine a starting point associated with a relative location on the substrate; and
directing the second imaging device to adjust the second viewing zone from the starting point to a target location on the substrate.

24. The method of claim 21, further comprising: directing the imaging device to adjust a viewing zone of the imaging device from the reference position to a target location.

25. The method of claim 23, wherein the target location is determined based on an input from a user.

26. The method of claim 23, wherein the target location is associated with an indexed position associated with a location of a sample on the substrate.

27. The method of claim 25, further comprising: receiving a second image of at least one of the encoded microstructures from the imaging device, and, based on the encoding formed in the encoded microstructures, identifying a movement of a sample disposed on the substrate.
